(19) United States
(12) Patent Application Publication
(10) Pub. No.: US 2024/0415907 A1
(43) Pub. Date: Dec. 19, 2024

(54) MIXTURE OF AT LEAST ONE BACTERIOPHAGE AND OF AT LEAST ONE YEAST AND METHOD FOR DRYING SAME

(71) Applicant: LESAFFRE ET COMPAGNIE, PARIS (FR)

(72) Inventors: Mickaël BOYER, MARCQ EN BAROEUL (FR); Alan ISRAEL, LILLE (FR); Edith POULAIN, LORGIES (FR); Jean-Bernard SOUICI, ESTAIRES (FR); Renaud TOUSSAINT, GONDECOURT (FR)

(73) Assignee: LESAFFRE ET COMPAGNIE, PARIS (FR)

(21) Appl. No.: 18/701,354

(22) PCT Filed: Oct. 14, 2022
(86) PCT No.: PCT/FR2022/051939

§ 371 Date: Apr. 15, 2024

(30) Foreign Application Priority Data
Oct. 15, 2021 (FR) FR2110984

(51) Int. Cl. A61K 36/064; A21D 8/04; A61K 9/16; A61K 35/76; C12C 12/00; C12G 1/00

(52) U.S. Cl. CPC A61K 36/064; A21D 8/047; A61K 9/1617; A61K 9/1623; A61K 9/1652; A61K 35/76; C12C 12/006; C12G 1/00; C12G 2200/05

(57) ABSTRACT

A method of manufacturing of a dry mixture of at least one yeast and/or yeast derivative and at least one bacteriophage, where the mixture being in the form of solid entities, and each solid entity is composed of at least one yeast and/or at least one yeast derivative and at least one bacteriophage and possibly at least one drying excipient, where the method is done by the mixing of at least one yeast and/or yeast derivative and at least one bacteriophage in suspension and the drying of this mixture. Also, a dry mixture of at least one yeast and/or yeast derivative and at least one bacteriophage, which is in the form of solid entities, where each solid entity is composed of at least one yeast and/or at least one yeast derivative and at least one bacteriophage and possibly at least one drying excipient and uses thereof.

### FIELD OF THE INVENTION

The present invention relates to a method of manufacturing of a dry mixture of at least one bacteriophage and at least one yeast and/or yeast derivative, a mixture comprising solid entities each of which consists of at least one bacteriophage and at least one yeast and/or yeast derivative and various uses of such a mixture.

### DESCRIPTION OF RELATED ART

Humans as well as animals or plants can play the role of host for bacteria giving rise to bacterial type infections. The use of bacteriophages was selected for training illnesses or digestive disorders of bacterial origin. For example, the document US20190255122 describes a method for treating or preventing a gastrointestinal inflammation or pain in a human comprising the oral administration of a composition comprising one or more bacteriophages selected from bacteriophages from the Siphoviridae family or Myroviridae family. In particular, the one or more bacteriophages are selected from LH01-Myoviridae, LL5-Siphoviridae, T4D-Myoviridae and LL12-Myoviridae. The bacteriophages could therefore play a prebiotic role serving to protect the gastrointestinal microflora.

Moye et al. (A Bacteriophage Cocktail Eliminates Salmonella typhimurium from the Human Colonic Microbiome while Preserving Cytokine Signaling and Preventing Attachment to and Invasion of Human Cells by Salmonella In Vitro. J Food Prot. 2019 August; 82 (8): 1336-1349) deals with bacteriophage cocktails administered to human patients in order to eliminate Salmonella strains from the intestine without disrupting the native microbiota. This cocktail also serves to prevent the risk of invasion of the intestinal epithelium by Salmonella. The bacteriophage cocktail studied in that article was prepared from three phage preparations sold by Intralytix, Inc.: ListShield™, EcoShield PX™ and SalmoFresh™.

Dissanayake et al. (Bacteriophages Reduce Pathogenic Escherichia coli Counts in Mice Without Distorting Gut Microbiota. Front Microbiol. 2019 Sep. 10; 10:1984) uses the same bacteriophage cocktail against the Escherichia coli O157/H7 strain. At the outcome of that study, the researchers concluded that such a cocktail had an antibiotic effect similar to that of ampicillin without showing an effect as harmful for the intestinal microbiota as ampicillin.

Further, it is known that the yeasts have a beneficial role both for human nutrition and health, and for animal nutrition and health or plant nutrition and health. For example, some strains of Saccharomyces cerevisiae are considered as probiotic yeasts promoting intestinal health. As seen above, the bacteriophages may for their part be used in order to prevent or treat a bacterial infection in humans, animals or plants. It is appropriate therefore to combine at least one bacteriophage and at least one yeast in order to prevent or treat bacterial infections while also allowing the yeast to develop and play their nutritional, protective and stimulating role in human, animal or plant health.

An attractive innovative concept consists of combining probiotic yeast and phages in a single product in order to combine their respective effects during the application (antibacterial effect of the phages and protection offered by the yeasts). For example, the document US20180161382 deals with compositions comprising at least one type of bacteriophage as a prebiotic agent and at least one probiotic agent which could be selected from Saccharomyces boulardii or Saccharomyces cerevisiae. The bacteriophage has a role in promoting the development of beneficial bacteria by reducing the populations of harmful bacteria and by releasing nutriments in its environment intended to be used by beneficial bacteria of an individual's digestive system. The listed prebiotic and probiotic agents are added separately within the composition. According to this document, each bacteriophage is specific to one undesirable bacteria. Consequently, as such, the bacteriophages do not directly affect any other organism in the digestive tract nor any probiotic. Thus, specific undesirable bacteria would be broken down and their cellular material would be available as nutriments for the probiotic or endogenous organism. Further, by weakening the population of specific undesirable bacteria, the probiotic organisms may successfully compete and establish a colony producing an environment suitable for them but inhospitable for the undesirable organism.

The presentation of microorganisms in dry form enhances handling, stability and long-term storage and the possibility of being used in gel caps or other forms of presentation and doses suited for specific applications (animal feed, food stuffs, etc.).

Various methods may currently be used for drying living microorganisms, but not all of them make it possible to get a satisfactory final viability level. The existing methods include for example freeze-drying, spray drying and fluidized air bed drying.

The applicant has extensive knowledge of the methods for obtaining yeast in dried form: quick-frozen intermediate-moisture yeast, active dry yeast (ADY) and instant dry yeast (IDY).

A sample method for drying of living microorganisms is given in the document FR 2,708,621 in which bacteria are co-dried with yeast. This co-drying is possible because bacteria have complex walls that are insensitive to break down by yeast proteases.

Bacteriophages are viruses which infect bacteria. They have walls made up solely of proteins, walls which are thinner and more fragile than those of bacteria. Thus, when it involves drying a suspension of bacteriophages (one family or a mixture of phages), in most of the cases, it is necessary to implement supports to enhance the survival and/or to obtain a final dry product having the required properties (shape, granule size distribution, dry extract, porosity, solubilization or instantaneousness property, compressibility, etc.). In fact, it is impossible to dry in this state because the amount of dry material in the suspension (coming from a lysed bacterial culture or saline solution suited to the storage of the phages) is generally very low (<5%). Drying under these conditions would not be economically attractive and especially too deleterious for the phages.

Thus, the drying support is intimately linked to the formulation concept which involves implementing one or more ingredients or supports or excipients with the microorganism so that the drying is possible and easy.

Further, and on the condition of having bacteriophages in powder form, the mixture of yeasts and bacteriophages in a finished industrial product would require overcoming some difficulties such as guaranteeing the homogeneity of the mixed powder or filing gel caps or other forms for presentation and dosing with a limited volume. In order to avoid problems connected with a powder form, it is possible to imagine a mixture of phages with yeasts in the form of yeast cream or pressed yeast. However, yeast cream and pressed yeast are acid (5.8 pH) and could be associated with a protease activity which could change both the phage titer and the lytic activity of the phages on the bacterial target.

Finally, the phages are biological entities requiring protection against the stress generally encountered during storage and according to their use (application on an inert or living support, in protected atmosphere or open air, ingestion by a human or an animal, etc.).

Thus, the prevention of bacterial infections by means of bacteriophages may be rendered complicated by the difficult conditions met in the animal or human stomach (pH of about 2 to 3), and also by exposure to bile and digestive enzymes in the gastrointestinal tract, which make the phages inactive. Similarly, the brief persistence of phages in various vegetable environments remains a major concern in biological control using phages directed against plant pathogenic agents. Indeed, UV radiation from sunlight may inactivate the phage during storage and interfere with the potential application thereof as a biological control agent.

It is also known that fermented foods and beverages based on fresh yeast may also be subject to contamination by bacteria. As seen above, bacteriophages may be used to fight against these bacteria. Among these foods and beverages, breadmaking products and wine or beer type fermented beverages can be listed.

Similarly, during production of bioethanol, more specifically first-generation bioethanol, it is necessary to control the natural flora of lactic bacteria whose proliferation could negatively affect the production yield. Therefore, the use of bacteriophages could serve to control lactic bacterial flora. Bioethanol corresponds to the ethanol produced by fermentation of agricultural products containing fermentable sugars.

There is therefore a need for mixtures of yeasts and bacteriophages having a resistance to pH variations and UV radiation while assuring the activity of the yeasts or yeast derivatives and the lytic activity of bacteriophages.

A phage is a virus which infects a bacteria. According to the invention, the terms “phage” and “bacteriophage” are interchangeable.

### BRIEF SUMMARY OF THE INVENTION

The invention aims to improve the situation.

According to a first aspect, an object of the present invention is a method of manufacturing of a dry mixture of at least one yeast and/or yeast derivative and at least one bacteriophage, said mixture being in the form of solid entities, each solid entity consisting of at least one yeast and/or at least one yeast derivative and at least one bacteriophage and possibly at least one drying excipient, wherein said method is characterized in that it is done by the mixing of at least one yeast and/or yeast derivative and at least one bacteriophage in suspension and the drying of this mixture.

According to a second aspect, an object of the present invention is a dry mixture of at least one yeast and/or yeast derivative and at least one bacteriophage, characterized in that it is in the form of solid entities, where each solid entity consists of at least one yeast and/or at least one yeast derivative and at least one bacteriophage and possibly at least one drying excipient.

Preferably, said mixture is obtained by the method according to the first aspect.

According to a third aspect, an object of the present invention is a use of the dry mixture according to the second aspect or of the dry mixture obtained from the method according to the first aspect as medicament in a composition intended to reduce the acidity of the gastric fluid.

According to a fourth aspect, an object of the present invention is a use of the dry mixture according to the second aspect or of the dry mixture obtained from the method according to the first aspect in a composition for plant stimulation, protection, biocontrol and/or nutrition.

According to a fifth aspect, an object of the present invention is a use of the dry mixture according to the second aspect or of the dry mixture obtained from the method according to the first aspect in a food composition or in a food supplement.

According to a sixth aspect, an object of the present invention is a use of the dry mixture according to the second aspect or of the dry mixture obtained from the method according to the first aspect in brewing and/or winemaking.

According to a seventh aspect, an object of the present invention is a use of the dry mixture according to the second aspect or of the dry mixture obtained from the method according to the first aspect in breadmaking.

According to an eighth aspect, an object of the present invention is a use of the dry mixture according to the second aspect or of the dry mixture obtained from the method according to the first aspect in the production of bioethanol.

### DETAILED DESCRIPTION OF THE INVENTION

Thus, according to a first aspect, an object of the present invention is a method of manufacturing of a dry mixture of at least one yeast and/or yeast derivative and at least one bacteriophage where said method is characterized in that it is done by mixing at least one yeast and/or yeast derivative and at least one bacteriophage in suspension and drying of this mixture.

Said mixture is in the form of solid entities, and each solid entity is composed of at least one yeast and/or at least one yeast derivative and at least one bacteriophage and possibly at least one drying excipient.

The term “composed of” is read as “consisting of.”

Thus according to a first aspect, an object of the present invention is a method of manufacturing of a dry mixture of at least one yeast and/or yeast derivative and at least one bacteriophage, the mixture being in the form of solid entities, and each solid entity consists of at least one yeast and/or at least one yeast derivative and at least one bacteriophage and possibly at least one drying excipient, where said method is characterized in that it is done by the mixing of at least one yeast and/or yeast derivative and at least one bacteriophage and the drying of this mixture.

Such a method allows production of stable formulations containing phages assuring optimal protection thereof and routing thereof towards action sites, such as the gastrointestinal tract or the phyllosphere of plants (aerial parts). The dried forms are preferred because of the easy handling thereof and long-term storage stability thereof, for example at ambient temperatures, thus avoiding the need for a cold chain for storage.

The yeasts or yeast derivatives, used as probiotics in animal, human and plant health were characterized for tolerance thereof and resistance thereof to several stress factors associated both with production methods (upstream processes, for example drying, storage) and stressful conditions of the intestinal tract (gastric pH, bile and digestive enzymes).

The combination of yeast and/or yeast derivative and phage constitutes an alternative for protecting the phages by the yeast or derivatives thereof and transporting them to the target site. Thus, the yeasts (or derivatives thereof) have a double role: on the one hand they play the probiotic role and, on the other, they serve as a support during the drying step, as shown by the following examples. This solution serves to maximize the beneficial effects of each of the components of the combination.

Further, and in particular in the case of the use for protection of plants, the mixture resulting from the method of co-drying phage with yeast and/or yeast derivatives serves to improve the protection of the phages against environmental conditions and UV effects (in vitro conditions), while also retaining the beneficial activity and properties of the yeast.

Such a mixture serves to reduce the costs associated with the use of compositions comprising both yeasts or yeast derivatives and bacteriophages by optimizing the management of inventory and supply thereof. It is therefore ready to use, reduces the risks of loss and does not lead to extra costs related to the mixing of dry yeast or yeast derivatives with bacteriophages, all while avoiding the risks of handling errors in the management of the microorganisms, like for example dosing errors. Indeed, these errors may occur in particular during the preparation of mixtures of yeast powders when the user must add the ferments in specific weight proportions. The use of this mixture makes it easier to prepare compositions involving the use of yeasts or yeast derivatives.

Another notable advantage of this method compared to a conventional dry/dry mixture between at least one yeast and one bacteriophage lies in the homogeneity of the resulting mixture. That way, the risk of loss of homogeneity is greatly minimized. This is because each solid entity composing the dry mixture resulting from the method according to the invention simultaneously comprises yeasts or yeast derivatives and bacteriophages. This dry mixture also serves to limit the handling of powdered products and therefore to limit the health risks. In other words, a single dry mixture is used.

Thus, this new approach of mixing the yeasts with the bacteriophages before drying thereof serves to improve performance, eliminate risks of handling errors related to the use of several powders or microorganisms on a single site, improves practicality and simplification, reduces costs, greatly limits losses following order changes and market fluctuations.

The method according to the invention may be implemented by using active yeasts and/or yeast derivatives.

The term “active yeast,” which is synonymous with “live yeast” or “fresh yeast,” designates a population of yeast cells that are metabolically active. When yeasts called “fresh” are used, activity designates the viability thereof.

A yeast derivative according to the invention is defined as a fraction obtained during the breakdown of the yeast by physical or chemical action, for example by plasmolysis, hydrolysis or autolysis of the yeast. The yeast derivatives are all products that could all be obtained from whole yeast cells or cells fractionated by physical or chemical action. In particular, they comprise yeast extracts obtained by autolysis or autolysates, yeast hulls, mannoproteins, inactivated yeasts. They most often come in more or less fine powder form after milling or in suspension in a rehydration medium, under yeast cream or pressed yeast form. Advantageously, the yeast derivative is the yeast hull or the yeast extract. Even more advantageously, the yeast derivative is the yeast hull.

The hulls may be obtained or prepared according to techniques known to the person skilled in the art, in particular by enzymatic lysis or by mechanical lysis (separation, concentration, etc.).

In one embodiment, the hulls are produced by enzymatic lysis (autolysis by their own proteolytic enzymes or heterolysis) of yeast cells following the separation of soluble and insoluble parts, for example by physical means, like centrifuging, and recovery of the insoluble part. The insoluble part is typically recovered by elimination of the soluble part by centrifuging. The insoluble part corresponds to the yeast hulls. The soluble part resulting from this method, with light color and low turbidity, is called yeast extract.

Preferably, the method of manufacturing of a dry mixture of at least one yeast and/or yeast derivative and at least one bacteriophage is characterized in that it comprises:
providing at least one yeast and/or yeast derivative, preferably in cream form, and at least one bacteriophage in suspension;
mixing said yeast and/or yeast derivative with said at least one bacteriophage so as to form a mixture;
performing a drying step of the mixture so as to form a dry mixture;
recovering the dry mixture.

Preferably, the bacteriophages are in suspension in an aqueous solution, preferably saline. The person skilled in the art will know how to adapt such a solution according to their needs, for example by choosing a buffered saline solution.

According to some embodiments, the drying step is done by freeze drying or spray drying or on a fluidized air bed.

Preferably the dry matter content of yeast and/or yeast derivatives within the mixture before the drying step of the at least one yeast or one yeast derivative and the at least one bacteriophage is located between 20 and 60% relative to the total quantity of dry matter in the mixture to be dried.

Preferably, the drying step may be preceded by a dehydration step to increase the dry matter content. This dehydration step is then followed by an actual drying step to obtain the final dry mixture to be recovered. In other words, the drying step could be done in two stages.

Preferably, the drying step may be followed by a step wherein the dry mixture is further divided, for example by milling.

Freeze-drying serves to obtain a lyophilizate which can be milled in flake form or fine powder whereas spray drying serves to obtain a finely divided dry product.

The principle of spray drying is to dehydrate the liquid droplets in a flow of hot gas (air for example) which circulates in the drying tower. The liquid to be dried (solution or suspension or mixture, with a dry extract adapted for not being too viscous) is nebulized in the form of fine droplets using a spraying device (nozzle or turbine) which is generally placed in the top of the tower. It involves drying by entrainment; the droplets are nearly instantaneously transformed into solid particles which are separated from the air at the end of drying in order to get a fine powder or a micro-granulated powder, depending on the configuration of the dryer (simple, double or multiple effect).

While the air inlet temperatures are generally high, for example between 100 and 300° C., preferably between 120 and 250° C., the outlet air temperature and especially the temperature of the product inside the tower is several tens of degrees lower because the particles, which are surrounded by a water film, cool during the change of state from liquid water to vapor. The person skilled in the art will know how to adapt these temperatures based on their needs.

Nonetheless even if the temperature of the product is lower, this technique may be destructive for the dehydration of living products like microorganisms. It is however possible to partially preserve the viability by making use of suitable and more moderate operating conditions (in particular addition of a drying support or additives in the formulation of the initial mixture and choice of the temperature scale). Furthermore, the transit time of the particles in the drying tower may also impact the viability; it will therefore be necessary to minimize it by taking care to target a final moisture content of the powder which is compatible with the desired lifetime of the product (storage).

Spray drying of the suspension of phages alone as it is produced (bacterial lysate) is not possible because the dry extract is too weak and the operation would be economically unattractive but could also be harmful for the microorganisms (use of high air temperatures). In order to increase the dry extract, according to the invention, yeast and/or yeast derivatives are used, and possibly secondary ingredients or excipients having a specific effect (e.g. protective).

Specific attention will be given to the dry extract targeted by the preparation to be dried: the liquid itself must not exceed a certain viscosity in order to be able to be pumped and transformed into fine droplets by the spraying device.

The preparation of the mixture is done until the ingredients or supports are completely dissolved or dispersed and this will be dried as quickly as possible in the spraying tower in order to avoid any breakdown of the product or microbial proliferation. Preferably, the mixture will be held at a low temperature throughout the drying time. The person skilled in the art will know how to choose the necessary temperature.

The drying parameters are adapted according to the configuration of the tower and the spraying device but also the properties of the mixture (viscosity, dry extract), where the purpose is to get a fine powder with a 10% maximum final moisture content, preferably 8% maximum, more preferably 6%.

In addition to yeasts and/or yeast derivatives, additional drying excipients may be listed, such as maltodextrin, native starch, trehalose and L-leucine.

Generally, freeze-drying is a drying technique allowing desiccation under vacuum of liquid or semi-pasty products which were previously frozen. This technique is often used for fragile products which do not stand direct drying; it thus allows assuring the stability of perishable products, stopping metabolism of biological products and obtaining easily rehydratable powdered products. Thus, a freeze-dried product has a high affinity for the solvent that it contains (generally water).

From a practical perspective, this operation comprises three major phases and thus one can speak of a freeze-drying cycle.

The first phase is an operation of freezing the product which serves to solidify the matrix and especially to crystallize the water that it contains in ice form. To do that the temperature of the product needs to be lowered sufficiently below the temperature of complete solidification thereof. The person skilled in the art will know how to choose the necessary temperature.

The second phase is a primary drying or sublimation step. During this phase, it is necessary to lower the pressure in the freeze-drying chamber, therefore to make a high vacuum. Thus, the pressure should be less than the vapor pressure of ice at the temperature under consideration. Further, the temperature of the product should remain below the initial melting temperature.

The third phase is a secondary desiccation step which serves to finish the dehydration by eliminating the final traces of water by desorption. It is characterized by the lowest possible pressure in the chamber and by a high product temperature which still remains below the denaturation temperature thereof.

Thus with this final phase, dry products having a very low residual moisture content (for example <1%) can be obtained.

Lastly, the associated operations of the freeze-drying cycle are:
preparing the product to be freeze-dried which generally consists of combining a mixture of excipients or supports with protective and cryoprotective effect;
protecting the final freeze-dried product which is often unstable and may quickly take up the solvent that it contained (hygroscopicity for an aqueous product) because of the highly porous structure thereof. In this case, it involves isolating the lyophilizate from the external environment and packaging it in a suitable way.
the phage suspension is formulated with adjuvants or excipients which will have a support and/or cryoprotection agent role. The dry extract (for example around 25 to 30%) needs to be increased in order to concentrate the phage suspension and thus reduce the quantity of water to be eliminated. According to the invention, this increase in dry matter is made possible by mixture with yeast and/or yeast derivatives. The person skilled in the art will know how to choose “bacteriophage/yeast and/or yeast derivative” ratio in order to get the best viability of the phages after drying.
the preparation of the mixture is done until complete dissolution or dispersion of the excipients and/or the yeast and/or the yeast derivative.

In addition to yeasts and yeast derivatives, other drying excipients may be listed, such as maltodextrin, native starch, trehalose and L-leucine.

During this step, the mixture will be completely cooled (for example <8° C.) and distributed in trays or vials while keeping a certain layer height (for example 15 mm), before undergoing freezing at below −20° C. in a freezer or deep freezer.

After checking the solidification of the mixture, which should be complete (water entirely crystallized), the containers or vials are placed on the shelves of the freeze dryer previously cooled by starting up the cold trap (for example −55° C.).

The person skilled in the art will know how to implement the desiccation steps and adjust the temperatures as a consequence.

At the end of the freeze-drying cycle, the lyophilizate generally has the appearance of a porous meringue. According to the case, this meringue is reduced to a fine powder by soft milling and preferably this operation will be done in a hygrometrically controlled enclosure (in order to avoid it taking up water again), before rapid packaging under vacuum or under inert atmosphere.

Preferably, the method according to the invention further comprises a step of extrusion of the dehydrated mixture so as to form an extruded mixture, where the drying step is done on the extruded mixture in a fluidized bed so as to form a dry mixture.

Drying in fluidized bed makes it possible to obtain a dry product in granular or vermicelli shape.

In general, the principle of drying in fluidized bed is to dehydrate the moist solid particles in a flow of hot air. In this case, these solid moist particles are obtained after a granulation step which serves to produce a solid having a suitable shape and density for fluidization in air. The particles (also called granules) are thus found suspended in hot air without touching, and it is their entire contact surface with the air which may be uniformly dried. The dry granules are characterized by a residual moisture content below 10%, preferably 8%, also preferably 5%, which provides a good stability of the product over time (storage).

The advantage of this technique is being able to perform moderate drying at low temperature which makes this a choice method for drying living microorganisms or fragile biological products. The size of the particles to be dried, after the granulation step, is important and the smaller they are the faster the product will dry. The time of exposure of the product to heat will also be reduced, which will favor a better viability level after drying.

From an application perspective, this technique is commonly used for drying baker's yeast and in this case instantaneous dry yeast is obtained in porous granular form which can be rehydrated very quickly in water or in a powder mixture (flour) to which water is added.

Granulation, also called extrusion, is the step preceding drying in fluidized bed and can only be done on pasty or semi-pasty products having a dry extract compatible with this operation of passing through an extruder. For example, filtration of a yeast cream gives pressed yeast which has this property.

Indeed, the mass to be dried is shaped in an extruder which produces thin continuous noodles, which are then broken up in short vermicelli to obtain the granules. If the mass to be extruded is too moist, the filaments tend to stick together after extrusion and it will no longer be possible to dry them in individualized form: at the end of drying, large aggregates will result which will retain some moisture.

The person skilled in the art will know how to adapt the moisture content of the mass to be extruded according to the needs.

For example, a way to counter this difficulty is the addition of a drying excipient. Preferably the drying excipient is selected from maltodextrin, native starch, trehalose and L-leucine.

Preferably, the drying step is done in the presence of the drying excipient, preferably maltodextrin.

The yeast may come from a strain selected from the species Saccharomyces cerevisiae and Saccharomyces boulardii, preferably the yeast comes from a strain selected from the strain Saccharomyces cerevisiae deposited on Oct. 17, 2007 under number CNCM I-3856, the strain Saccharomyces cerevisiae deposited on Mar. 22, 2018 under number CNCM I-5298, the strain of Saccharomyces boulardii deposited on Aug. 21, 2007 under number CNCM I-3799, the strain Saccharomyces cerevisiae deposited on Aug. 31, 2016 under number CNCM I-5129, the strain Saccharomyces cerevisiae deposited on Aug. 31, 2016 under number CNCM 1-5130 or the strain Saccharomyces cerevisiae deposited on Feb. 9, 2011 under number CNCM I-4444.

Preferably, the yeast comes from a strain selected from the species Saccharomyces cerevisiae and Saccharomyces boulardii, preferably the yeast comes from a strain selected from the strain Saccharomyces cerevisiae deposited on Oct. 17, 2007 under number CNCM I-3856, the strain Saccharomyces cerevisiae deposited on Mar. 22, 2018 under number CNCM I-5298, and the strain of Saccharomyces boulardii deposited on Aug. 21, 2007 under number CNCM I-3799.

Preferably, the one or more bacteriophages are selected from those having an antibacterial activity against bacterial strains selected from Escherichia coli, Listeria monocytogenes, Campylobacter jejuni, Staphylococcus aureus, Clostridium perfringens or strains of the Salmonella genus, and lactic bacteria. Lactic bacteria may be selected from: Lactobacillus fermentum (new taxonomy: Limosilactobacillus fermentum), Lb. delbrueckii, Lb. Reuteri (Limosilactobacillus reuteri), Lb. Casei (Lacticaseibacillus casei), Lb. Brevis (Levilactobacillus brevis), Lb. Perolens (Schleiferilactobacillus perolen), and L. amylovorus. Antibacterial activity is understood to mean a lytic activity by the bacteriophage on the bacteria following infection of the bacteria thereby.

“Lactic bacteria” is understood to mean gram-positive bacteria, anaerobes partially tolerant to oxygen and capable of fermenting sugars into lactic acid.

Known bacteriophages which could be used are selected from T4 sold by DSMZ under catalog number DSM 4505 and belonging to the family of Myoviridae, T5 sold by DSMZ under catalog number DSM 16353 and belonging to the family of Siphoviridae or T7 sold by DSMZ under catalog number DSM 4623 and belonging to the family of Podoviridae, or a mixture thereof or from the SalmoFresh™ or FOP™ phage mixtures sold by Intralytix Inc. The bacteriophages T4, T5 and T7 have an antibacterial activity against Escherichia coli. The cocktail of six bacteriophages belonging to the Myoviridae family sold under the name SalmoFresh™ has an antibacterial activity against the pathogenic strains of the Salmonella genus, for example Salmonella enterica or even Salmonella typhimurium, Salmonella Heidelberg, Salmonella Newport, Salmonella Kentucky, Salmonella infantis. FOP™ is a unique and exclusive mixture of 15 individual lytic phages which offer broad protection against pathogenic strains of Salmonella enterica, Escherichia coli and Listeria monocytogenes.

According to a second aspect, the invention relates to a dry mixture of at least one yeast and/or yeast derivative and at least one bacteriophage, characterized in that it being in the form of solid entities, where each solid entity is composed of at least one yeast and/or at least one yeast derivative and at least one bacteriophage and possibly at least one drying excipient.

The term “composed of” is read as “consisting of.”

Thus according to a second aspect, the invention relates to a dry mixture of at least one yeast and/or yeast derivative and at least one bacteriophage, characterized in that it being in the form of solid entities, where each solid entity consists of at least one yeast and/or at least one yeast derivative and at least one bacteriophage and possibly at least one drying excipient.

Preferably, the mixture is obtained according to a method described according to the first aspect.

Preferably, the yeast derivatives are yeast hulls.

Preferably, the dry mixture is divided in powder form which comprises solid entities and, possibly a drying excipient.

Preferably, the solid entities have the shape of flakes, grains, vermicelli or granules.

Preferably the drying excipient is selected from maltodextrin, native starch, trehalose and L-leucine.

Preferably, the yeast and bacteriophages are selected from those previously described.

According to a third aspect, an object of the present invention is a use of the dry mixture according to the second aspect or of the dry mixture obtained from the method according to the first aspect as medicament in a composition intended to reduce the acidity of the gastric fluid.

According to a fourth aspect, an object of the present invention is a use of the dry mixture according to the second aspect or of the dry mixture obtained from the method according to the first aspect in a composition for plant stimulation, protection, biocontrol and/or nutrition.

More specifically, this is for the treatment or protection of plants against diseases produced or provoked by pathogenic agents, in particular fungal, bacterial or viral, for the induction or stimulation of natural defenses in a plant against pathogenic agents.

According to a fifth aspect, an object of the present invention is a use of the dry mixture according to the second aspect or of the dry mixture obtained from the method according to the first aspect in a food composition or in a food supplement.

According to a sixth aspect, an object of the present invention is a use of the dry mixture according to the second aspect or of the dry mixture obtained from the method according to the first aspect in brewing and/or winemaking.

According to a seventh aspect, an object of the present invention is a use of the dry mixture according to the second aspect or of the dry mixture obtained from the method according to the first aspect in breadmaking.

According to an eighth aspect, an object of the present invention is a use of the dry mixture according to the second aspect or of the dry mixture obtained from the method according to the first aspect in the production of bioethanol.

### MATERIALS AND METHODS

T4 Bacteriophage or T4 Phage belonging to the Myroviridae family which have a long contractile tail

T5 Bacteriophage or T5 Phage belonging to the Siphoviridae family which have a long non-contractile tail

T7 Bacteriophage or T7 Phage belonging to the Podoviridae family which have a short non-contractile tail

These three phages are lytic phages which infect Escherichia coli.

SalmoFresh™ is a unique and exclusive mixture of six individual lytic phages which provide a broad protection against pathogenic strains of the Salmonella genus, for example Salmonella enterica or even Salmonella typhimurium, Salmonella Heidelberg, Salmonella Newport, Salmonella Kentucky, Salmonella infantis.

FOP™ is a unique and exclusive mixture of 15 individual lytic phages which offer broad protection against pathogenic strains of Salmonella enterica, Escherichia coli and Listeria monocytogenes.

The list of microorganisms used is given in table 1 below.

TABLE 1

| Names | Type | Reference |
|---|---|---|
| T4 Phage | Bacteriophage | DSMZ 4505 |
| T5 Phage | Bacteriophage | DSMZ 16353 |
| T7 Phage | Bacteriophage | DSMZ 4623 |
| SalmoFresh ™ | Bacteriophages | Mixture of 6 phages |
| | | produced by Intralytix |
| | | targeting pathogenic strains of the |
| | | Salmonella genus |
| FOP ™ | Bacteriophages | Mixture of 15 phages |
| | | produced by Intralytix |
| | | targeting Salmonella enterica, |
| | | Escherichia coli |
| | | and Listeria monocytogenes |
| Salmonella | Bacteria | SN388 |
| enterica | | |
| Escherichia coli | Bacteria | Escherichia coli |
| | | DSMZ 613 (Migula 1895) |
| | | Castellani and Chalmers 1919 |
| Escherichia coli | Bacteria | Escherichia coli 342-5 |
| Listeria | Bacteria | LM114 000689 |
| monocytogenes | | |
| Saccharomyces | Yeast | CNCM I-4444 (deposited Feb. 9, |
| cerevisiae | | 2011) |
| Saccharomyces | Yeast | CNCM I-3856 (deposited Oct. 17, |
| cerevisiae | | 2007) |
| Saccharomyces | Yeast | CNCM-I-3799 deposited |
| boulardii | | Aug. 21, 2007 |

The list of reagents used is given in table 2 below.

TABLE 2

| Name: | Reference | CAS Number |
|---|---|---|
| Sodium chloride | Merck 31434 | 7647-14-5 |
| Magnesium sulfate heptahydrate | Merck M2773 | 10034-99-8 |
| Cycloheximide 1% | | 66-81-9 |
| Potassium phosphate, monobasic | Merck P0662-500G-M | 7778-77-0 |
| Tris-HCl 1M (pH 7.5) | Merck T2319 | |
| Gelatin, 2% | Merck G1393 | 9000-70-8 |
| Hydrochloric acid, 37% | Sigma 320331-500 ml | |
| Sodium hydroxide | Merck 30620-1KG-M | |
| Yeast extract | Procel 351 pw | |
| Trypto caseine soy broth | bioMérieux 51019 | |
| Trypto caseine soy agar | bioMérieux 51044 | |
| Agar | BD 214530 | |
| Difco YM Agar | BD 271210 | |
| Pork bile extract | Sigma B88631-100G | 8008-63-7 |
| Pancreatin from pig pancreas | Sigma P1750-100G | 8049-47-6 |
| Pepsin 500 U/mg | Sigma 77160-100G | |

### Simulated Gastric Fluid (SGF)

Simulated gastric fluid is prepared according to the work of Ma et al. (2008), Colom et al. (2015) and Vinner et al. (2018) (for 100 mL):
0.2 g NaCl
0.4 g pepsin (500 U/mg)
distilled water QS 100 mL
pH adjusted with HCl or NaOH, line of pH prepared: 2.5, 3.0, 3.5, 4.0

The enzymatic activities (U/mL) were reproduced according to the work of Adouard et al. 2019. Once prepared, the liquid is preheated to 37° C.

For the co-dried samples, 50 mL of SGF is poured in a 180 mL jar and 0.5 g of the sample is added, which corresponds to a dilution of 1E-02 (1 in 100).

For the phages in solution (controls), 9.9 mL of SGF is poured in a 60 mL jar in which 100 μL of phage solution is added, which corresponds to a dilution of 1E-02; the phage solution is diluted to obtain a phage concentration closest to that of the co-dried sample.

The jars are then incubated at 37° C. with stirring (100 rpm) to mimic the passage through the stomach. At time zero (right after adding the sample) and at 15, 30, 60, 120 minutes phage counts by spots according to the method described below are done without forgetting to stir the jar before collecting the aliquot for dilution.

Yeast counts are also done in the gastric liquid at pH 2.5 at the beginning (TO) and end (T120) of the experiment according to the method described.

The pH is measured again at the end of the experiment.

Simulated Intestinal Fluid (SIF) Simulated intestinal fluid is prepared according to the work of Ma et al.

(2008), Colom et al. (2015) and Vinner et al. (2018) (for 100 mL):
0.68 g KH2PO4
1 g of pork bile
2.16 g of pancreatin
distilled water QS 100 ml
pH adjusted to 6.8 with NaOH

The enzymatic activities were reproduced according to the work of Adouard et al. 2019. Once prepared, the liquid is preheated to 37° C.

For the co-dried samples, 50 mL of SIF is poured in a 180 mL jar into which 0.5 g of sample is added, which corresponds to a dilution of 1E-02.

For the phages in solution (controls), 9.9 mL of SIF is poured in a 60 mL jar in which 100 μL of phage solution is added, which corresponds to a dilution of 1 in 100; the phage solution is diluted to obtain a concentration of phages the closest possible to the concentration of the co-dried sample. The jars are then incubated at 37° C. with stirring (100 rpm) to mimic the passage through the intestine. At time zero (right after adding the sample) and at 30, 60, 120 minutes (Minekus et al. 2014) phage counts by spots according to the method described below in paragraph 141 are done without forgetting to stir the jar before collecting the aliquot for dilution.

Yeast counts are also done at the beginning (TO) and end (T120) of the experiment according to the method described.

The pH is measured again at the end of the experiment.

Counting of co-dried phages and yeasts

The counting of phages co-dried with the yeast is done after a step of rehydration of the powder in the Stomacher blender and are done according to a method adapted from an internal procedure: Count of bacteriophages per PFU Rehydration of the co-dried samples
weigh 1 g of co-dried phage-yeast sample using a precision balance and place it in a Stomacher bag;
add 9 mL of sterile distilled water at 37° C. to the bag and then place it in the Stomacher blender for 3 minutes at medium speed.

Technique for in-depth counting of the phages
melt the TSA+YE gelatin (Trypto casein soy agar (TSA)+Yeast Extract (YE) at 6 g/L of agar+cycloheximide (0.5%)) and keep it above melting;
prepare 90 mm Petri dishes with about 15 mL of TSA+YE+cycloheximide (0.5%) medium, allow them to dry under hood for 30 minutes;
seed 20 mL of TSB+YE (trypto casein soy broth (TSB)+yeast extract (YE)) broth from a colony coming from a fresh culture;
incubate under stirring until obtaining a culture at the beginning of exponential growth phase (example: stop the culture at a DO included between 0.2 and 0.7 for E. coli, Salmonella and Listeria);
read the DO value.

Prepare decimal dilutions of the rehydrated co-dried sample in the Eppendorf tubes containing 900 μL of the SM.

In a 13 mL tube containing 100 μL of host bacterial solution (DO included between 0.2 and 0.7), at 100 μL of the desired phage dilution and leave in contact for 10 minutes.

Add 3 mL of TSA+YE with 6 g/L of agar+cycloheximide medium and pour on a previously poured and dried gelatin dish. Allow to solidify.

Incubate at the growth temperature of the host bacteria for 24 hours.

### Technique for Counting of the Phages by Spots

### Preparation of Agar Dishes

melt gelatin at 6 g/L agar and then add 0.5% cycloheximide, hold it above melting
seed 20 mL of TSB+YE broth from a colony coming from a fresh culture;
incubate under stirring until obtaining a culture at the beginning of exponential growth phase (example: stop the culture at a DO included between 0.2 and 0.7 for E. coli, Salmonella and Listeria); Prepare square Petri dishes with 25 mL of TSA+YE+cycloheximide (0.5%) medium, allow them to dry under hood for 30 minutes;
read the DO value.
add 9 mL of TSA+YE with 6 g/L of agar+cycloheximide medium to a 13 mL tube containing 300 L of host bacteria solution (DO included between 0.2 and 0.7) and pour on a previously poured and dried gelatin dish.
allow to solidify.

### Prepare Decimal Dilutions of the Phage Solution

fill the columns of wells of 96-well microplates with 180 μL of SM buffer by starting at column 3 and by skipping every other column in order to allow spot seeding.
in the wells of the first column add 100 μL of solution coming from rehydration of the sample which here constitutes the first dilution at 1 in 10 (1E-01).
using a multichannel pipet, collect 20 UL in the first column and transfer it into the wells of the following dilution column and so on until the last dilution.

### Deposit on Dish

From one microplate line (96 wells), 10 μL needs to be collected with a suitable multichannel pipette.

Next, divide the square Petri dish previously seeded with the host bacteria in four columns and deposit the 10 μL spots of phage/yeast solutions, allow them to dry and incubate at the growth temperature of the host bacteria for 24 hours.

The counting of yeasts is done in parallel.

After the sample is rehydrated, 100 μL is collected to perform decimal dilutions in Eppendorf tubes filled with 900 μL of sterile distilled water.

100 μL of the dilution of interest are collected, placed on YM gelatin dish and smeared.

The dishes are then incubated for three hours at 25° C.

Independent of the technique, the results are expressed as follows:

Theoretical titer PFU/g DM (before drying): This titer is calculated from the phage assay titer for the phage suspension and from the actual composition of the formulation achieved in the laboratory which considers all the ingredients added (e.g. yeast, fraction, protective agents, additives). To avoid biases, this titer is referred to the dry matter of the mixture (before drying).

Assay titer PFU/g DM (after drying): This is the actual titer as it is assayed from the dry finished product and referred to the actual extract of the dry finished product.

Losses of phages (Log 10 PFU/g) are equal to the difference between:

Log 10 (theoretical titer PFU/g DM before drying)−Log 10 (assayed titer PFU/g DM after drying) and expressed in Log 10 PFU/g.

### Tests of Gastric Resistance

Simplified in vitro gastrointestinal resistance tests were done on co-dried samples. The goal of these tests is to analyze the survival of phages and yeasts by counting at different points over time in the simulated gastrointestinal fluids. These tests repeat the counting method described above without the rehydration phase with the Stomacher mixer which is done here directly in the simulated liquid.

0.5 g of sample is placed in 50 mL of fluid. A line of pH from 2.5 to 4.0 was prepared for the gastric test.

### Alkalinization Potential

For the purpose of defining what excipient is responsible for the increase of gastric pH, co-dried samples were produced by freeze-drying the SalmoFresh™ solution with each phage excipient individually in the same proportions (99% DM excipient versus 1% DM phage). The samples are made in the laboratory.

0.5 g of each sample was placed in contact with 50 mL of simulated gastric fluid at pH 2.5. After homogenization, the pH of each preparation was measured on the pH meter after homogenization.

### UV Resistance Tests

UV resistance tests were done on rehydrated co-dried samples for three minutes with a Stomacher blender (1 g in 9 mL of sterile distilled water) and on the phages in solution in the UV BS-03+UV-MA chamber with UV-C bulbs. The measured irradiation is 12 mW/cm². For each sample form, three identical aliquots are made in order to be exposed for three different times (5, 15, 30 minutes). These tests are adapted following the tests done by the Ramirez et al. 2018 team. The survival of the phages and the yeasts is measured by the counting method.

The counting at TO is identical for the co-dried sample and for the rehydrated form thereof; it is done according to the method of spots described above; the counting of the phages in solution is done according to an internal procedure.

For the co-dried samples, about 1.5 g is weighed and spread in an open Petri dish at the center of the chamber. After UV exposure, 1 g is weighed by precision balance and then rehydrated three minutes in a Stomacher blender so that the phages are counted according to the method of spots described above.

For the rehydrated co-dried samples, 1 mL is placed in an open Petri dish at the center of the chamber. After exposure to UV, 100 μL is collected for counting the phages according to the method of spots described above.

For the phages in solution, 1 mL is placed in an open Petri dish at the center of the chamber. The phage concentration of the solution is the closest possible to the phage concentration in the co-dried sample by dilutions. After exposure to UV, 100 μL is collected for counting the phages according to an internal procedure.

The counting of yeast is done at TO and at T30 according to the method described.

### Stability Tests

The stability of the phage-yeasts co-dried products stored in packets or pills under vacuum was studied over three months under three temperature and humidity conditions:
25° C./60% RH
30° C./65% RH
40° C./60% RH

Counts of phages according to the in-depth counting technique are done at different points in time: 0, 15, 30, 60, 90 days. The a w (water activity) is measured at 25° C. at TO, 30 and 90 days by means of an AquaLab Series 4TEV dewpoint hygrometer.

The water activity is an important parameter related to the quality of the dry products. The aw values are an indicator of the microorganisms possibility of growth and the production of toxins. The value of the water activity varies between 0 (dry product to the point that all the water is bound to the product, and therefore without reactive quality) and 1 (pure water, without solute).

Indeed, the aw indicates the share of free water in a product; meaning available for example for growth of microorganisms. The higher the aw, the more water is available for the development of these microorganisms. Water activity values over 0.6 could support the growth of microorganisms.

### EXAMPLES

### Example 1: General Method for Obtaining a Dry Yeast-Bacteriophage Mixture by Drying in Fluidized Air Bed

### The Steps of the Method:

verifying the pH of the yeast cream or yeast derivatives and possibly adjusting the pH to 7.0 with 10% sodium hydroxide
filtering the yeast cream over a plate filter under pressure: obtaining pressed yeast (PY)
mixing of the PY with the phage suspension and the desiccant or protective agents (also called drying excipients)
granulating and shaping by extrusion
drying of granules in a fluidized air bed dryer
verifying final moisture content of the dried granules and packaging under vacuum or under inert atmosphere

Thus, 16.5 g of the suspension of SalmoFresh™ phages (0.6% dry matter), 2 g trehalose dihydrate, 2 g maltodextrin and 0.065 g L-Leucine are added to 200 g pressed yeast (30% dry extract). After having mixed for one minute in a Matfer AlphaMix 5 L mixer beater equipped with a mobile dasher for mixing, 60 g starch (potato starch) is added and the mixture is mixed for one minute. The mixed mass containing about 40% dry extract is then extruded on DRC i10 extruder equipped with an extrusion grid with 1 mm openings. The resulting filaments are collected in a 5 L beaker and manually broken up by hard shaking. The drying is done on a Fluid Bed Dryer Tornado M501 (Sherwood) equipped with a multi-chamber support.

2×80 g of damp granules are placed in two glass drying chambers (60 mm diameter and 400 mm length) equipped on their base with a 250-mesh stainless steel sieve (fluidization air inlet). The drying program is started and comprises two steps, each carried out at a temperature between 4° and 60° C. The fluidization and heating air is dehydrated by an air dehumidifier (Munters ComDry M190Y).

The drying is finished when the dry extract of the granules reaches at least 94%. The packaging of the granules is done under vacuum in order to assure the best stability of the product over time.

### Example 2: General Method for Obtaining a Dry Yeast-Bacteriophage Mixture by Spray Drying

### The Steps of the Method:

preparing the mixture by adding the yeast or yeast derivatives and, optionally, drying ingredients in the phage suspension
vigorous mixing until complete dissolution or dispersion of all ingredients
verifying the pH of the mixture and possibly adjustment to 7.0 with 10% sodium hydroxide
drying of the mixture in a spraying tower
verifying the final moisture content and packaging under vacuum or under inert atmosphere of the fine powder

Thus, a suspension of yeast hulls is partially reconstituted by dispersing 75.3 g of Safmannan® yeast hulls in 336 g of demineralized water (preparation 1). The pH of the yeast hull suspension could be adjusted to 7.0 with 10% sodium hydroxide. The preparation 1 is cooled to a temperature included between 2 and 10° C.

Next, 42 g of trehalose dihydrate and 2.7 g of L-leucine are dissolved in 112 g of warm demineralized water (preparation 2). After cooling of preparation 2, 39 g of the suspension of SalmoFresh™ phages (2.5% dry extract) are added and mixed under vigorous stirring. Then, this mixture is added to preparation 1 under vigorous stirring in order to obtain the mixture to be dried comprising the phages, yeast or yeast derivatives and the various supports or ingredients. This mixture is held under stirring at a low temperature throughout the length of the process.

The drying is done on a Mini Spray Dryer B-290 (Büchi) tower equipped with a compressed air bifluid nozzle, a peristaltic pump, a cyclone for separation of dry particles from damp air and a polyester outlet filter. The equipment is supplemented by an air dehumidifier (Munters ComDry M190Y) which processes the air at the inlet to the spraying tower.

The operating parameters (air temperature and flow rate, feed rate of the solution to be dried, etc.) are adjusted for drying the phages under moderate conditions by exposing them to moderate temperatures; the outlet temperature is therefore checked for not exceeding 60° C.

The drying is done when the moisture content of the powder is below 6%. The spray dried powder is packaged under vacuum.

### Example 3: General Method for Obtaining a Dry Yeast-Bacteriophage Mixture by Freeze-Drying

The steps of the method:
preparing the mixture by adding yeasts or yeast derivatives and, optionally, other ingredients in the phage suspension
vigorous mixing until complete dissolution or dispersion of all ingredients
verifying the pH of the mixture and possibly adjustment to 7.0 with 10% sodium hydroxide
rapid cooling and freezing to at least −20° C.
drying of the frozen mixture in a freeze dryer under reduced pressure
milling of the lyophilizate and verification of the final moisture content
packaging under vacuum or under inert atmosphere of the finely divided dry product.

Thus, the pH of a freshly collected yeast cream (21.3% dry extract) is adjusted to 7.0 by 10% sodium hydroxide. A protective solution comprising the supports or excipients is prepared: 18.9 g of trehalose dihydrate, 18.9 g of maltodextrin and 0.63 g of L-leucine are dissolved in 51.1 g of warm demineralized water. 7.9 g of the suspension of T5 phages (2.3% dry extract) is added to this protective solution, after cooling thereof. The mixture is held under stirring and cooled. This mixture is added to 113 g of the preceding yeast cream, still under stirring and cooled. It contains about 27.4% dry extract.

The liquid yeast/phage/protective agent mixture is distributed in glass trays or vials such that the height of the layer in the container does not exceed 15 mm.

All the containers are cooled and frozen quickly in the freezer to a temperature≤−20° C.

After freezing for several hours, the containers are put in a laboratory freeze dryer (Lyovapor L-200 Büchi) whose freeze-drying shelves and chamber have been previously cooled.

As previously discussed, a primary desiccation followed by a secondary desiccation is started and the freeze-drying process is stopped at the end of 80 to 96 hours. The lyophilizates are finely and gently reduced to powder using a mortar mill, and possibly passed through a 500 μm mesh sieve. The final moisture content is verified and must be <3%. Packaging under vacuum is done quickly for avoiding alteration of the product over time.

The mixtures used in the tests described below were dried according to one of the three methods described in Examples 1-3.

In the context of the stability tests, the counts were done with the in-depth counting technique for phages. In the context of other tests, the counting was done with the counting by spots technique and with a starting sample which constitutes the 1E-02 dilution; the detection limit of this method is therefore 4 Log (named below ND).

When confronted with acidic conditions, the phages seem to suddenly become inactive below a certain threshold which is located around pH 3.5 (Ma et al. 2008, Davis et al. 1985). A line of four pH values (2.5, 3.0, 3.5, 4.0) was therefore tested for encompassing this threshold and analyzing the full spectrum that might be found in a fasting stomach or after absorption of an alimentary bolus (gastric resistance test in gastric fluid).

Whatever the drying mode used, the T5 bacteriophages or the SalmoFresh™ bacteriophage cocktail retained an entirely satisfactory activity for industrial use. Analogous results were obtained with T4 and T7 bacteriophages and the FOP cocktail.

### Example 4: Gastric Resistance Test in Gastric Fluid with a Mixture Obtained from Co-Drying by Spraying of a S. cerevisiae CNCM I-3856 Yeast or Yeast Hulls with the T5 Bacteriophage or with the SalmoFresh™ Bacteriophage Cocktail

| pH | 0 | 30 min | 120 min | Time and mixture tested |
|---|---|---|---|---|
| 2.5 | ND | ND | ND | Log PFU/g yeast CNCM |
| 3 | 6.6 | 5.58 | <4 | I-3856 + T5 |
| 3.5 | 6.73 | 6.72 | 6.09 | bacteriophage |
| 4 | 6.55 | 6.67 | 6.55 | |
| 2.5 | ND | ND | ND | Log PFU/g T5 |
| 3 | ND | ND | ND | bacteriophage alone |
| 3.5 | ND | ND | ND | |
| 4 | 6.28 | 6.15 | 4.88 | |

The phage alone in solution is inactivated under pH 4 whereas it is possible to count the phages co-dried with the yeast starting at pH 3.

At pH 2.5, none of the tested phage forms remained active.

At pH 3, the T5 phages co-dried with the yeast CNCM I-3856 remained active for at least 30 minutes before slipping below the detection threshold.

At pH 3.5, after two hours, a loss of T5 phages co-dried with the yeast CNCM I-3856 is seen as greater Log PFU/g.

At pH 4, the T5 phages co-dried with the yeast CNCM I-3856 are in a more stable environment. It should be noted that the concentration in PFU/g of the samples may increase between TO and T30. This is most certainly due to the fact that the powder (in particular the spray dried powder) can form agglomerates which do not always dissolve very quickly.

Yeasts from the samples placed in contact with the gastric fluid at pH 2.5 were counted at the beginning (TO) and at the end (T120) of the experiment. The results are similar for all tests: after two hours in an acid environment, the yeast concentration is very slightly reduced.

| Time | 0 | 120 min |
|---|---|---|
| Log CFU/g | 9.01 | 8.91 |

### Example 5: Gastric Resistance Test in Gastric Fluid with a Mixture Obtained from Co-Drying by Spraying of a Yeast with the SalmoFresh™ Bacteriophage Cocktail

| pH | 0 | 30 min | 120 min | Time and mixture tested |
|---|---|---|---|---|
| 2.5 | 6.19 | 5.11 | <4 | Log PFU/g yeast CNCM |
| 3 | 7.72 | 7.59 | 5.70 | I-3856 + SalmoFresh ™ |
| 3.5 | 7.95 | 8.08 | 7.63 | bacteriophage cocktail |
| 4 | 8.03 | 8.03 | 7.92 | |
| 2.5 | ND | ND | ND | Log PFU/g |
| 3 | ND | ND | ND | SalmoFresh ™ |
| 3.5 | 5.90 | 5.31 | ND | bacteriophage cocktail |
| 4 | 7.53 | 6.80 | 6.19 | alone |

Upon comparing the gastric resistances of phage solutions alone (T5 and SalmoFresh™), a better survival was observed with phages present in the SalmoFresh™ solution. It is also noteworthy that the SalmoFresh® solution contains a phage cocktail and that the sensitivity to pH of each phage may be different.

Note that co-drying by spraying with yeasts allows phages to remain active for 30 minutes even at pH 2.5 before slipping below the detection threshold.

At pH 3.0, 3.5 and 4.0, the samples remain active during the two hours of the experiment.

Similarly, yeasts from the samples placed in contact with the gastric fluid at pH 2.5 were counted at the beginning (TO) and at the end (T120) of the experiment. The results are similar for all tests: after two hours in an acid environment, the yeast concentration is very slightly reduced.

| Time | 0 | 120 min |
|---|---|---|
| Log CFU/g | 9.75 | 9.46 |

### Example 6: Gastric Resistance Test in Gastric Fluid with a Mixture Obtained from Co-Drying of Yeast Hulls with the T5 Bacteriophage or with the SalmoFresh™ Bacteriophage Cocktail

| | | | | | Time and mixture |
|---|---|---|---|---|---|
| pH | 0 | 30 min | 60 min | 120 min | tested |
| 2.5 | ND | ND | ND | ND | Log PFU/g yeast hulls + |
| 3 | 7.7′ | 7.60 | 6.57 | ND | T5 bacteriophage |
| 3.5 | 8.02 | 8.33 | 8.19 | 7.81 | |
| 4 | 8.23 | 8.43 | 8.34 | 8.19 | |
| 2.5 | ND | ND | ND | ND | Log PFU/g yeast hulls + |
| 3 | 7.09 | 8.34 | 7.79 | 6.78 | SalmoFresh ™ |
| 3.5 | 8.67 | 8.29 | 7.53 | 7.25 | bacteriophage cocktail |
| 4 | 9.09 | 8.83 | 8.40 | 7.41 | |

These samples were made by co-drying by spray drying. The survival of the phages seems identical for pH 3.0, 3.5 and 4.0 despite an acidity neutralized in a more weakly way by the samples compared to the other spray-dried tests using the yeast CNCM I-3856.

It was observed at pH 3 that the T5 phages survive 60 minutes and the phages from the SalmoFresh™ solution survive throughout the 120 minutes of the experiment. The probable hypothesis is that since the powder resulting from these tests was even less soluble than the spray-dried powder obtained with the living yeasts, the diffusion of the phages occurred later, which could participate in the protection thereof. This hypothesis could also explain why more phages are counted after 30 minutes of exposure than the counting done at TO min.

### Example 7: Resistance Test in Intestinal Fluid with a Mixture Obtained from Co-Drying of Yeast with the T5 Bacteriophage or with the SalmoFresh™ Bacteriophage Cocktail

| | | | | | Time and |
|---|---|---|---|---|---|
| Drying | | | | | mixture |
| method | 0 | 30 min | 60 min | 120 min | tested |
| Control | 6.87 | 6.95 | 6.85 | 6.90 | T5 bacteriophage |
| Fluidized | 6.18 | 5.78 | 6.11 | 6.20 | Log PFU/g yeast |
| bed | | | | | L13 + T5 |
| | | | | | bacteriophage |
| Spray drying | 6.92 | 7.24 | 7.29 | 7.37 | Log PFU/g yeast |
| Freeze- | 7.58 | 7.66 | 7.68 | 7.75 | CNCM I-3856 + T5 |
| drying | | | | | bacteriophage |
| Control | 8.04 | 8.04 | 8.10 | 7.92 | bacteriophage |
| | | | | | cocktail alone |
| Fluidized bed | 6.56 | 6.39 | 6.36 | 6.48 | Log PFU/g yeast |
| Spray drying | 8.34 | 8.57 | 8.68 | 8.70 | CNCM I-3856 + |
| Freeze- | 8.01 | 8.05 | 8.15 | 8.19 | SalmoFresh ™ |
| drying | | | | | bacteriophage |
| | | | | | cocktail |

The intestinal resistance tests are based on the same principle as the gastric tests, except that only one pH is tested (pH 6.8). It can be seen from this that intestinal fluid has no impact on the survival of the phages or yeasts.

| Time | 0 | 120 min | Mixture tested |
|---|---|---|---|
| Log CFU/g | 8.79 | 8.70 | yeast CNCM |
| | | | I-3856 + T5 |
| | | | bacteriophage |
| Log CFU/g | 9.40 | 9.57 | yeast L47 + |
| | | | SalmoFresh ™ |
| | | | bacteriophage |
| | | | cocktail |

### Example 8: Potential Alkalinization Test

In order to determine what ingredients from the formulations had this alkalinization effect which serves to protect the phages in an acid medium, five samples were made all composed of 1% SalmoFresh™ phages in DM (dry material)/TDM (total dry material) and 99% of a single excipient used. 0.5 g of these samples were placed in contact with 50 mL of gastric fluid at pH 2.5. After dissolving the powders, the pH was measured.

The dried samples with yeast and L-leucine caused significant pH variations. With a 3.59 measured pH, the sample containing yeast even allowed exceeding the 3.5 inactivation threshold. It can be observed that it is the yeast which constitutes the most effective drying support and therefore which is present in significant quantity in the various formulations unlike the L-leucine.

| | | | pH after |
|---|---|---|---|
| | | | addition |
| | | | of |
| | | | 50 mL |
| | | | of SGF |
| | | | at pH |
| Drying | Dried phages | Formula | 2.5 |
| Freeze-drying | SalmoFresh ™ | L-leucine | 3.40 |
| | | Trehalose | 2.50 |
| | | maltodextrin | 2.48 |
| | | starch | 2.49 |
| | | yeast | 3.59 |

### Example 9: UV Radiation Resistance Test

| 0 | 5 min | 15 min | 30 min | Time and mixture tested |
|---|---|---|---|---|
| 6.22 | 5.06 | ND | ND | Log PFU/g T5 |
| | | | | bacteriophage alone |
| | | | | (control) |
| 6.54 | 6.51 | 5.90 | 5.55 | Log PFU/g yeast CNCM |
| 8.03 | | 8.03 | 7.92 | I-3856 + T5 |
| | | | | bacteriophage |
| 8.75 | ND | ND | ND | Log PFU/g |
| | | | | SalmoFresh ™ |
| | | | | bacteriophage cocktail |
| | | | | alone |
| 6.27 | 5.88 | 5.90 | 5.70 | Log PFU/g yeast CNCM |
| | | | | I-3856 + SalmoFresh ™ |
| | | | | bacteriophage cocktail |

| Time | 0 | 30 min | Mixture tested |
|---|---|---|---|
| Log CFU/g | 9.41 | 9.29 | yeast CNCM |
| | | | I-3856 + T5 |
| | | | bacteriophage |
| Log CFU/g | 9.50 | 9.57 | yeast CNCM |
| | | | I-3856 + |
| | | | SalmoFresh ™ |
| | | | bacteriophage |
| | | | cocktail |

The phages in solution and the phages co-dried with the yeast by spray drying were exposed to UV-C for 30 minutes with updates at 0, 5, 15, 30 minutes for counting the phages. The counting was done by spot as with the test for gastric resistance and with the same samples. Since the first dilution available was the 1E-01 dilution (coming from the rehydration with the Stomacher), the detection limit of the technique is 3 Log.

It can be observed that the phages in solution (T5 and SalmoFresh™ phages) are very sensitive to UV-C. The phages from the SalmoFresh™ solution are no longer active after five minutes of exposure and the T5 phages only last for five minutes. It is the opposite for the phages co-dried with the yeast which are only slightly affected by the UV-C.

The counting of the yeast before and after 30 minutes of exposure served to show that for all configurations tested, the UV-C exposure had no impact on their survival.

### Example 10: Temporal Stability Test

Stability tests over three months were done on the samples co-dried by spray drying at 25° C./60% RH, 30° C./65% RH and 40° C./75% RH. The aw (water activity) and the loss of phages were measured at the beginning (TO day), middle (T30 days) and end (T90 days) of the experiment.

| Time and | | | | |
|---|---|---|---|---|
| Conditions | 0 | 30 days | 90 days | Mixture tested |
| 25° C./60% | 8.35 | 7.24 | 6.63 | Log PFU/g S. boulardii + |
| RH | | | | T5 bacteriophage |
| 30° C./65% | 8.35 | 7.12 | 6.23 | |
| RH | | | | |
| 40° C./75% | 8.35 | 6.51 | 6.23 | |
| RH | | | | |
| 25° C./60% | 8.83 | 8.54 | 8.37 | Log PFU/g S. boulardii + |
| RH | | | | SalmoFresh ™ |
| 30° C./65% | 8.83 | 8.52 | 8.19 | bacteriophage cocktail |
| RH | | | | |
| 40° C./75% | 8.83 | 8.17 | 6.93 | |
| RH | | | | |

| | 0 | 30 days | 90 days | Time |
|---|---|---|---|---|
| 25° C./60% | 0.05 | 0.07 | 0.09 | a_{w} S. boulardii + T5 |
| RH | | | | bacteriophage |
| 30° C./65% | 0.05 | 0.06 | 0.08 | |
| RH | | | | |
| 40° C./75% | 0.05 | 0.07 | 0.18 | |
| RH | | | | |
| 25° C./60% | ND | 0.07 | 0.09 | a_{w} S. boulardii + |
| RH | | | | SalmoFresh ™ |
| 30° C./65% | ND | 0.06 | 0.08 | bacteriophage cocktail |
| RH | | | | |
| 40° C./75% | ND | 0.08 | 0.19 | |
| RH | | | | |

The very low values of aw indicate that the water part released in the resulting product is very low, sufficiently low for blocking the growth of microorganisms and therefore assuring the stability of the product over time.

1-20. (canceled)

21. A method of manufacturing of a dry mixture of at least one yeast and/or yeast derivative and at least one bacteriophage, where said mixture being in the form of solid entities, and each solid entity consists of at least one yeast and/or at least one yeast derivative and at least one bacteriophage and possibly at least one drying excipient, wherein said method is done by the mixing of at least one yeast and/or yeast derivative and at least one bacteriophage in suspension and the drying of this mixture.

22. The method according to claim 21, wherein said method comprises:
providing at least one yeast and/or yeast derivative, preferably in cream form, and at least one bacteriophage in suspension;
mixing said yeast and/or yeast derivative with said at least one bacteriophage so as to form a mixture;
performing a step of drying the mixture so as to form a dry mixture; and
recovering the dry mixture.

23. The method according to claim 21, wherein the drying step is done by freeze drying or spray drying or on a fluidized air bed.

24. The method according to claim 21, wherein the drying step is done in the presence of the drying excipient, preferably maltodextrin.

25. The method according to claim 21, wherein the yeast comes from a strain selected from the species Saccharomyces cerevisiae and Saccharomyces boulardii, preferably the yeast comes from a strain selected from the strain Saccharomyces cerevisiae deposited on Oct. 17, 2007 under number CNCM I-3856, the strain Saccharomyces cerevisiae deposited on Mar. 22, 2018 under number CNCM I-5298, and the strain of Saccharomyces boulardii deposited on Aug. 21, 2007 under number CNCM I-3799.

26. The method according to claim 21, wherein the one or more bacteriophages are selected from those having an antibacterial activity against bacterial strains selected from Escherichia coli, Listeria monocytogenes, Campylobacter jejuni, Staphylococcus aureus, Clostridium perfringens or strains of the Salmonella genus, lactic bacteria.

27. The method according to claim 21, wherein the yeast derivatives are yeast hulls.

28. A dry mixture of at least one yeast and/or yeast derivative and at least one bacteriophage, wherein the dry mixture is in a form of solid entities, where each solid entity consists of at least one yeast and/or at least one yeast derivative and at least one bacteriophage and possibly at least one drying excipient.

29. The dry mixture according to claim 28, wherein the solid entities have the shape of flakes, grains, vermicelli or granules.

30. The dry mixture according to claim 28, wherein the yeast comes from a strain selected from the species Saccharomyces cerevisiae and Saccharomyces boulardii, preferably the yeast comes from a strain selected from the strain Saccharomyces cerevisiae deposited on Oct. 17, 2007 under number CNCM I-3856, the strain Saccharomyces cerevisiae deposited on Mar. 22, 2018 under number CNCM I-5298, and the strain of Saccharomyces boulardii deposited on Aug. 21, 2007 under number CNCM I-3799.

31. The dry mixture according to claim 28, wherein the one or more bacteriophages are selected from those having an antibacterial activity against bacterial strains selected from Escherichia coli, Listeria monocytogenes, Campylobacter jejuni, Staphylococcus aureus, Clostridium perfringens or strains of the Salmonella genus, lactic bacteria.

32. The dry mixture according to claim 28, wherein the yeast derivatives are yeast hulls.

33. The dry mixture according to claim 28 for use as a medicament.

34. A method of treating acidity of gastric fluid in a subject, comprising administering to a subject in need there of an effective amount of the dry mixture according to claim 28.

35. A composition for treatment of acidity of gastric fluid comprising the dry mixture according to claim 28.

36. A composition for the stimulation, protection, biocontrol and/or nutrition of plants comprising the dry mixture according to claim 28.

37. A food composition or a food supplement comprising the dry mixture according to claim 28.

38. A method of beer making and/or winemaking comprising adding the dry mixture according to claim 28.

39. A method of breadmaking comprising adding the dry mixture according to claim 28.

40. A method of producing bioethanol comprising adding the dry mixture according to claim 28.
